(19) United States
(12) Patent Application Publication
(10) Pub. No.: US 2023/0058370 A1
(43) Pub. Date: Feb. 23, 2023

(54) SECURING DEVICE AND ASSEMBLY COMPRISING A RETAINING ELEMENT AND A SECURING DEVICE

(71) Applicant: EBNET MEDICAL GMBH, Schwerin (DE)

(72) Inventor: Jens EBNET, SCHWERIN (DE)

(21) Appl. No.: 17/767,212

(22) PCT Filed: Oct. 2, 2020
(86) PCT No.: PCT/EP2020/077673

§ 371 Date: Apr. 7, 2022

(30) Foreign Application Priority Data
Oct. 10, 2019 (DE) 10 2019 127 310.2

(51) Int. Cl. A61M 25/02

(52) U.S. Cl. CPC A61M 25/02; A61M 2025/0286; A61M 2025/028

(57) ABSTRACT

The invention relates to a securing device (1) designed for securing a medical, cosmetic, decorative or other type of item to the skin of a living being, wherein the securing device (1) has at least one main puncturing element (2) with a puncturing tip (3), wherein the main puncturing element (2) is designed for penetrating into the skin of the living being, and the securing device (1) has at least one securing section (6, 16) coupled to the main puncturing element (2) and for securing the item to be secured on the securing device (1), wherein the the securing device (1) is designed for securing the main puncturing element (2) in the skin by means of a rotational movement of the securing device (1), whose axis of rotation runs orthogonal to the surface of the skin.

The invention relates to a securing device which is designed for securing a medical, cosmetic, decorative or other article to the skin of a living being, wherein the securing device has at least one skin piercing element having a piercing tip, wherein the skin piercing element is designed to penetrate the skin of the living being, and the securing device has at least one securing portion which is coupled to the skin piercing element and is designed for securing the article that is to be secured on the securing device. The invention also relates to an assembly comprising a retaining element and at least one securing device of the type explained above.

An important field of use of the securing device according to the invention is in the medical sector, for example for securing catheters, tubes and drains, and corresponding systems and medical products (hereinafter only “catheters”) or parts thereof to the skin or to other anatomical structures (hereinafter only “skin”) of a living being. Where the word “patient” is used in the following, this means explicitly both a patient in the field of human medicine and one in the field of veterinary medicine, that is to say a living being in the broadest sense.

Catheters, which are inserted through the skin and the subcutaneous connective tissue into the target structures of a body, have to be regularly secured to the skin so that they do not slip out of the target structure or the body. A variety of influences make this securing difficult, of which some examples are: sweat and secretion production by the skin, escape of blood and secretion from the piercing site, and the need for skin care. The securing of catheters to the skin, for example especially also vascular catheters, therefore represents a particular challenge.

In practice, the challenge is mainly overcome by suturing the catheters to the skin. Further securing is generally provided by plasters, which additionally have the function of providing a sterile covering of the piercing site or of the entry point of the catheter into the skin. Securing by plasters or also by dressing material alone is usually insufficient.

The suturing of catheters is associated with a number of disadvantages and dangers to the patient and user. For example, when securing a catheter with a suture there is always the danger of the needle and thread penetrating too deeply into the body and, for example, harming a blood vessel. This commonly occurs in practice and is manifested, for example, by an immediate escape of blood from the skin along the suture and/or by the formation of a bruise near the suture site.

In addition, the suturing or knotting technique has to be learned and practiced: tied knots can come undone and loosen. Moreover, the suture or the knot must be neither too tight (possible reduced blood flow to the skin region due to compression of small blood vessels) nor too loose (insufficient securing of the catheter in the desired position). There is a considerable outlay in terms of material, since the needle, thread, and possibly instruments such as a needle holder for guiding the needle and a scalpel for severing the thread after the suturing, must be provided in a sterile state and have to be disposed of or recycled in a complicated manner. In addition, the choice of the appropriate thread is a not a trivial one, since the thread has to have certain properties.

Sometimes, a plurality of threads are used. These threads must be easy to remove after the catheter is withdrawn. Moreover, it is sometimes necessary for the position of the catheter to be changed (e.g. drawn back) in a controlled manner after it has been put in place. This may require removal of stitches and renewed suturing. The suturing (and sometimes also the removal of stitches) is also frequently painful when the patient is conscious. In addition, a securing procedure requires the needle to at least be passed in and out of the skin, and this sometimes requires local anesthetic, which may also cause complications.

Moreover, the passage of the needle in and out of the skin always results in the formation of at least two potential ports of entry for microorganisms into the body. The thread can in this case also serve as a type of “guide rail” for the admission of microorganisms. This is of practical importance in the case of patients who are seriously ill or whose immune systems are weakened. Not least, the user may suffer a needle stick injury during the suturing procedure and, for example, become infected with a potentially life threatening viral disease. The patient and assistants may also suffer a needle stick injury.

A securing device of the generic type is known from U.S. Pat. No. 4,683,895.

The object of the invention is to improve the securing of such articles to the skin of a living being in order to avoid the disadvantages mentioned at the beginning.

This object is achieved in the case of a securing device of the type mentioned at the beginning in that the securing device is designed for securing the skin piercing element in the skin by a rotational movement of the securing device, the axis of rotation of which runs orthogonally to the skin surface. The invention has the advantage that the securing of the article that is to be secured to the skin can be considerably more simple and, moreover, more secure against inadvertent loosening of the securing. The insertion of the securing device in the skin can be carried out very simply by the user, specifically with a simple rotational movement by which the securing device is simply “screwed” by its skin piercing element into the skin, similarly to how a screw is screwed into a workpiece. In addition, the securing device can be removed again in a similarly simple manner from the living being by it being unscrewed from the skin in the opposite direction of rotation.

For the improved securing of the skin piercing element in the skin, the skin piercing element can be formed with a roughened surface and/or with small barbs.

The securing device according to the invention is suitable potentially for all situations in which an article has to be rapidly, securely and simple secured to the skin.

The securing device according to the invention can be combined with all known and future medical products, even with all catheters and drain tubes. Prior to the use, the skin piercing element or the entire securing device can be surrounded by a removable protective cap.

The securing device can be designed in such a manner that even a comparatively small rotation, i.e. a small angle of rotation about the axis of rotation, is sufficient for the securing to the skin, for example an angle of rotation in the range of 150° to 360°, wherein, in certain embodiments of the securing device, even a larger angle of rotation of the rotational movement may be expedient for securing the securing device to the skin, for example a plurality of full revolutions, e.g. up to three revolutions.

The securing portion can be mounted foldably or pivotably on the securing device. Depending on the embodiment of the securing device, the latter can have one securing portion or a plurality of securing portions. The features mentioned in conjunction with the securing portion therefore apply both to one securing portion and to a plurality of securing portions.

The skin piercing element penetrates into the skin of a living being only very superficially and to an already defined maximum penetration depth. The securing device according to the invention can therefore be an optimum compromise between all known types of securing. A required securing in the skin tissue takes place, but without having a completely adverse effect on the barrier function of the skin.

The article that is to be secured can be configured as a separate component from the securing device. This means that the article that is to be secured is basically obtainable independently of the securing device, or, in other words, the securing device can be provided separately from the article that is to be secured. The article that is to be secured can then be fastened to the securing portion of the securing device and released again therefrom at any time.

The skin piercing element can in each case have different diameters or diameters which change over the longitudinal extent. For example, it is possible for the skin piercing element to be configured with a smaller diameter from the root region in the direction of the piercing tip and with a larger diameter in the direction of the coupling portion. These properties moreover make it possible for the piercing pain to be reduced since the required piercing channel in the skin is expanded slowly. Furthermore, those portions of the skin piercing element which are located deeper in the living being have less potential for injuring deeper anatomical structures, such as blood vessels and nerves. This is an important safety aspect. In addition, in the correctly secured state on the living being, a diameter of the skin piercing element that increases from the piercing tip in the direction of the root region of the skin piercing element provides optimum sealing of the piercing channel towards the environment.

An advantage of the configuration of the skin piercing element is furthermore that effective tunnelling of the piercing channel is achieved on a small area. If, nevertheless, deeper anatomical structures are intended to be achieved, the risk of, for example, skin microorganisms penetrating therein is potentially reduced since they have to cover a longer distance and have to first propagate through the tunneled and convoluted piercing channel. The risk of damage to the catheter by the piercing point is also reduced by the controlled and locally limited use of the skin piercing element.

The skin piercing element can be configured to be firmer or less firm. The “resilient” properties can thus be configured to be weaker or greater. It is thereby ensured that, on securing, e.g. even in the event of swelling in the region of the skin, the contact pressure does not become too great and the securing element as a whole is adapted to the individual circumstances at the securing site.

It is also possible for the main piercing element to be connected in an angular and/or rotatable manner to a further component of the securing element, for example to the coupling portion. Use even on uneven or angled surfaces is thereby possible.

Up to now, use is sometimes also made of dressings applied in circular form around extremities for securing catheters, which dressings may, inter alia, lead to an impairment of the arterial blood flow or to a venous blockage and may also be heavily soiled. In addition, the dressings may restrict patient comfort and mobility, and the piercing site cannot always be checked for infectious complications since it cannot be seen.

Patients not capable of cooperating may inadvertently remove the dressings. Relatively large skin regions may be damaged by plaster devices, and plaster allergies also occur. Moreover, in practice, although plasters may be loosened from a patient's skin, they continue to remain persistently stuck to the catheter. This is unhygienic and attempts to remove a plaster from a catheter may lead to damage and even to inadvertent perforation and cutting through of same if, for example, sharp objects, scissors or scalpels are used.

Sometimes, plasters for securing catheters also have to be adapted individually in size, which can signify a considerable outlay in terms of staff and time. Not least, the suturing procedure of catheters may cause the latter to be damaged by the suture. In such a case, the catheter has under some circumstances to be completely changed. These disadvantages can be overcome by using the securing device according to the invention. If, however, in accordance with current guidelines, additional securing aids, such as plasters or dressings, should be required, the required aids may, of course, also be used when the securing device according to the invention is used. Use can even be made of a spray-on plaster in order to additionally seal the piercing site. Threads can also nevertheless be used and, for example, guided through the securing portion.

The piercing tip can also be configured in various variants to be beveled. In particular, it can also be configured or provided with a polished section in such a manner that punching out and displacing skin particles into deeper anatomical structures is made difficult or impossible.

The piercing tip may be composed of a material ensuring that the piercing tip becomes blunt when subjected to body heat. Further injuries by the tip are therefore prevented. The remaining portion of the skin piercing element can be composed of a non-thermoplastic material which remains hard even at body temperature. The configuration of the skin piercing element in the form of a spiral spring causes the latter to remain securely secured in the skin even when the piercing tip is blunt.

According to an advantageous embodiment of the invention, it is provided that the securing device has a manual actuating element at which the securing device can be gripped manually and the rotational movement necessary for securing the securing device on the skin can be exerted on the securing device. This permits simple manual actuation and therefore simple use of the securing device by the user. The manual actuating element can be configured, for example, in a similar manner to a wing nut, or can be configured as a flat or differently shaped gripping element extending transversely with respect to the axis of rotation. According to an advantageous embodiment of the invention, the skin piercing element is coupled non-rotatably to the manual actuating element. Accordingly, a rotational movement exerted manually on the actuating element is transmitted substantially unchanged to the skin piercing element. The securing portion can be coupled non-rotatably to the skin piercing element or can be mounted rotatably in relation to the skin piercing element. The securing portion can be configured, for example, as a cutout or indentation on the manual actuating element.

According to an advantageous embodiment of the invention, it is provided that the securing device has a skin contact surface which comes into contact with the skin surface when the securing device is fully secured on the skin of the living being. This has the advantage that the penetration depth of the skin piercing element into the skin can be restricted by the skin contact surface. The skin contact surface can therefore form a stop when screwing the skin piercing element into the skin.

Malfunctions of the securing device are thereby avoided. The securing device is therefore particularly use-friendly, both for medical users and for the living being to which the securing device is fastened. In an advantageous embodiment, the skin contact surface can protrude over the skin piercing element in the circumferential direction, i.e. in a direction orthogonally to the axis of rotation.

The skin contact surface can have particular material properties, such as increased frictional resistance or a particular stickiness, for optimized securing of a retaining or securing element. Also conceivable, for example, are colloidal, pasty or gel-like properties of the skin contact surface in order to damage the skin as little as possible.

According to an advantageous embodiment of the invention, it is provided that the skin piercing element protrudes directly from the skin contact surface. This is required for a simple structural design of the securing device. The component of the securing device that has the skin contact surface can therefore be used directly for the arrangement and fastening of the skin piercing element.

By way of example, the securing device can be composed overall only of three elements, namely the skin piercing element, the skin contact surface and the manual actuating element if the latter has the securing portion. As will also be explained below, the securing device can also have further advantageous features.

The skin piercing element can be configured differently, for example as a screw-like structure.

According to an advantageous embodiment of the invention, it is provided that the skin piercing element is configured in the form of a spiral spring. This has the advantage that the engagement strength of the skin piercing element in the skin of the living being is minimized. In particular, more substantial injuries of the skin are avoided. Owing to its spiral spring form, the skin piecing element can be screwed helically like a thin curved needle into the skin. The securing device can be configured in such a manner that the longitudinal axis of the skin piercing element in the form of a spiral spring is aligned with the axis of rotation of the rotational movement necessary for securing the securing device to the skin. This permits simple operation of the securing device by the user.

The skin piercing element in the form of a spiral spring can have one or more turns. The one turn can extend over an angular range of 360° or also less than 360°, for example in the range of 150° to 360°. A plurality of turns can extend, for example, over an angular range of up to 3×360°.

According to an advantageous embodiment of the invention, it is provided that the turns of the skin piercing element in the form of a spiral spring are spaced apart from one another in the direction of the longitudinal axis of the skin piercing element in the form of a spiral spring. This permits simple and controlled screwing of the skin piercing element into the skin without excessive irritations or injuries to the skin occurring.

According to an advantageous embodiment of the invention, it is provided that the securing device has at least one stop by which the maximum angle of rotation of the securing device about the axis of rotation is limited. By this means, the securing device is configured to be particularly secure and use-friendly since the stop prevents it from being screwed too far into the skin.

The stop can interact with a travel limiting element of a retaining element, as is also described below. If the stop butts against the travel limiting element of the retaining element, the rotational movement of the securing device about the axis of rotation is stopped.

According to an advantageous embodiment of the invention, it is provided that the main piercing element in the form of a spiral spring has a pitch of less than 3 mm/360°. The pitch can also be less than 2 mm/360° or less than 1 mm 360°. In this way, the skin piercing element has a comparatively small pitch, and therefore the skin piercing element which is screwed into the skin can be securely retained even without particular locking measures. As a result, the securing device cannot be unintentionally loosened from the skin. It is advantageous here if the pitch of the skin piercing element is greater than 0.1 mm/360°. The pitch is understood here as meaning the penetration depth of the skin piercing element into the skin in millimeters (mm) based on an angle of rotation of 360°.

The object mentioned at the beginning is also achieved by an assembly comprising a retaining element and at least one securing device of the type explained above, wherein the retaining element has at least one cutout through which the securing device can be passed at least together with the skin piercing element, wherein the retaining element can be secured on the skin of the living being by means of the securing device by the skin piercing element that has been guided through the cutout being secured in the skin by a rotational movement of the securing device, the axis of rotation of which runs orthogonally to the skin surface. The retaining element can be fixable to the skin of the living being, for example, by means of the securing portion of the securing device. The advantages explained previously can also be realized by this means. The retaining element can form, for example, the medical, cosmetic, decorative or other article that is to be secured and that is to be fastened to the skin. The retaining element can also be a separate component from such an article that is to be fastened. For example, the retaining element can be a securing tab of a catheter, via which the catheter according to the prior art can be firmly sutured to the skin. However, this is no longer required when the securing device according to the invention is used since the securing of the retaining element to the skin can be realized by screwing the securing device into the skin. The retaining element can also have, for example, a plurality of cutouts through which a respective securing device can be passed at least together with the skin piercing element. In this case, the retaining element can be secured in the manner explained above to the skin of the living being by means of the securing devices.

According to an advantageous embodiment of the invention, it is provided that the assembly has at least two securing elements or at least two skin piercing elements on the retaining element. By this means, the securing to the skin can be configured more securely and reliably. Loosening from the skin can be avoided even more reliably. In this case, the skin piercing elements can also have different properties, for example different material properties and/or diameters. A differentiation can also be made, for example, between skin piercing elements which are used for the securing and between those which are intended primarily to prevent rotational movements of the entire retaining element. The latter skin piercing elements may be configured, for example, to be smaller, i.e. atraumatic. Said assisting skin piercing elements may optionally then also be rectilinear. An undesirable rotational movement of the retaining element or of the securing device can already be prevented.

According to an advantageous embodiment of the invention, it is provided that the securing device has a coupling portion which is arranged in the longitudinal direction of the securing device between the securing portion and the skin piercing element. In this way, the securing device is somewhat extended in the longitudinal direction such that it can protrude through the retaining element or the cutout thereof and can nevertheless secure the retaining element on the side facing away from the skin of the living being. If the securing device has a plurality of securing portions, the coupling portion can be arranged between the skin piercing element and the first fixing portion, as seen from the skin piercing element.

According to an advantageous embodiment of the invention, it is provided that the coupling portion can be guided into the cutout and is mounted rotatably in the cutout. In this way, the coupling portion together with the inner circumference of the cutout can form a rotational mounting for the securing device on the retaining element.

According to an advantageous embodiment of the invention, it is provided that the coupling portion directly adjoins the skin piercing element in the longitudinal direction. The skin piercing element therefore protrudes from the coupling portion in the longitudinal direction. The skin piercing element can be coupled nonrotatably here to the coupling portion. The skin contact surface can be configured, for example, as a surface of the coupling portion that faces the skin.

According to an advantageous embodiment of the invention, it is provided that the securing portion or a further securing portion of the securing device protrudes over the coupling portion in the circumferential direction. Such a securing portion protruding over the coupling portion in the circumferential direction can be configured, for example, similarly to a screw head which protrudes above the corresponding screw hole in the circumferential direction. The securing portion does not have to protrude over the coupling portion over the entire circumference in the circumferential direction; it is sufficient if the securing portion protrudes over the coupling portion, for example, at two or three points on the circumference in the circumferential direction.

The securing device can be connected captively to the retaining element or can be coupled loosely to the retaining element. In the last-mentioned case, the connection between the securing device and the retaining element is produced only when the retaining element is to be directly secured or is already secured to the skin of the living being by means of the securing device.

If the securing device is coupled captively to the retaining element, it cannot be lost during the handling of the assembly with the retaining element and the securing device. Nevertheless, it may be possible, for example with increased application of force, to release the securing device again from the retaining element.

According to an advantageous embodiment of the invention, it is provided that the coupling portion has, on the outer circumference, at least one connecting element via which the securing device is fastenable to the retaining element and can be mounted rotatably on the retaining element. The previously mentioned captive fastening of the securing device to the retaining element can be realized via the connecting element. The connecting element can be configured, for example, as an external thread on the coupling portion or as a collar protruding over the coupling portion in the circumferential direction. Such a collar can surround the coupling portion over the entire circumference or at least in sections over the circumference.

According to an advantageous embodiment of the invention, it is provided that the retaining element has a fastening element which is complementary to the connecting element and which can be brought into engagement with the connecting element of the securing device in order to fasten the securing device to the retaining element and to mount same rotatably on the retaining element. This permits a simple and secure fastening of the securing device to the retaining element. The fastening element, which is complementary to the connecting element, can be configured, for example, as an internal thread, which corresponds to an external thread, on the inner circumference of the cutout of the retaining element. Depending on the embodiment of the connecting element, the complementary fastening element can also be configured as an internal groove in the cutout or as an edge region of the cutout, in particular as an edge region on that side of the retaining element which faces the skin.

According to an advantageous embodiment of the invention, it is provided that the securing device has at least one electrical insulating element by means of which an electrical current flow flowing via the securing device is prevented between an article secured on the securing portion and the skin of the living being. An undesirable current flow from the article that is to be secured into the patient or vice versa is thereby avoided. Accordingly, it can be ensured that burns or other irritations cannot undesirably occur on the skin of the patient due to certain operating techniques, such as, for example, coagulation. The securing device can have one or more electrical insulating elements. By way of example, an electrical insulating element can be arranged at any location between the securing portion of the securing device and the skin piercing element. Alternatively, the securing portion itself can also be configured to be electrically insulating or can be configured to be electrically insulating at least at the location at which the article that is to be secured comes into contact with the securing portion, for example by means of an electrically insulating material coating. The skin piercing element itself may also be composed of an electrically insulating material or may be coated at least on the outer surface with an electrically insulating material.

Further Possible Advantages of the Invention are:
The securing device according to the invention makes securing catheters to the skin much easier, quicker, safer and more cost-effective.
The design of the securing device according to the invention is of great relevance in practice. The intention in particular is also to permit catheters to be secured with less pain and more comfortably since the design means that the securing now takes place in a highly controlled and rapid manner.
Local numbing of the securing site can ideally be entirely dispensed with.
It is quick and simple to use. The securing device according to the invention can be operated intuitively; all of the components can be structurally integrated.
It is safer to use. The danger of an additional injury to user and patient (needlestick injury) is minimal or even eliminated.
It is cost-effective to use. The securing device according to the invention can be made available as a mass-produced item. Its use is therefore also advantageous from the financial point of view.
The use is also advantageous from an ecological aspect since no further waste is produced in the securing and, for example, a residual thread together with needle does not have to be thrown away or a sterile thread holder does not have to be recycled.
The securing device according to the invention also securely fastens the article that is to be secured even under difficult circumstances but can be quickly released again where necessary. An assistant is not required at any point.
The securing device affords the advantage that the contact pressure with which the securing device secures the article that is to be secured can be predefined within narrow limits. In this way, for example, there is less danger of a reduced blood flow to the skin region beneath the article that is to be secured. Since the article that is to be secured is not pressed with too great a force onto the skin, even during secure securing, small blood vessels are not appreciably compressed, and the flow of blood to the skin is thus maintained.
Hygienic aspects can be taken into account. The securing device according to the invention can be designed, for example, as a product that is to be used just once. The risk of contamination of the skin piercing element by the user is low since the securing device is gripped at the actuating element.
The securing device according to the invention is compatible with customary catheters and also with recently developed catheters.
The securing device according to the invention can already be a constituent part of a catheter or can be applied as an individual element to the catheter.
A variable length of the skin piercing element permits optimum use in different subgroups of living beings. For example, the skin piercing element for use in smaller living beings can be made shorter. The length of the skin piercing element can in particular also be adapted to use in different anatomical regions of a living being.
Customary catheters often already have holes for suturing. These holes are punched out on securing tabs, which protrude from the catheter to the left and right (“securing wings”). The securing device according to the invention is compatible with these and is therefore optimally adapted to the components of existing catheters.

The entire securing device or at least parts thereof, for example only the skin piercing element, can be made of antibacterial and/or antimicrobial material or can be coated with such a material. The entire securing device or at least parts thereof can be made of a material compatible with MRT/nuclear magnetic resonance tomography. The skin piercing element can in particular be made of a different material than the securing portion. The securing portion can be made, for example, of a plastic material, in particular a hard plastic.

The entire securing element or individual components of the securing element can be formed from transparent material so that the piercing site can be checked visually at any time for complications, for example infections. This material can be, for example, a hard plastic or a hard glass.

The skin piercing element can be made from metal or another material, for example likewise from a hard plastic. The skin piercing element can be made in particular also, at least partially, of an electrically non-conductive material. In particular wherever the skin piercing element is connected to at least one further component of the securing device, it can be connected to said further component via an electrically non-conductive material in order to achieve electrical insulation in relation to said further component. It is also possible for the skin piercing element to consist of a wire. This wire can be surrounded by a further material or can be coated on the inside and/or on the outside by such or embedded in such. The wire can be resistant to cutting and puncturing and can also be in the form of a wire mesh and reinforced by further structures. The wire can also be configured as a hollow wire.

It is also possible that the skin piercing element is made of a resorbable material which, through contact with the body, is wholly or partially broken down and/or dissolved within a defined time period. The material from which the skin piercing element is made can moreover have thermoplastic properties and/or properties that change according to the environment or active external influences. Thus, the material used can, through contact with the body, assume properties different than those when it is located outside the body. For example, the material can become softer or firmer when it comes into contact with the body. For example, the material can also form an increased frictional resistance or adhesive properties as soon as it comes into contact with the body. For example, the material can also change in shape as soon as it comes into contact with the body. It is advantageous, for example, if the material curves, i.e. has properties like a bimetal, and thus permits anchoring of the skin piercing element in the skin and in the subcutaneous connective tissue. It is also advantageous, for example, if the material changes in terms of its diameter, e.g. expands, when it comes into contact with the body. In this way, the skin piercing element can be fixed more firmly and more securely in the skin, or it fixes itself by virtue of these material properties. The material can also be provided with a surface coating which, through contact with the body, forms at least one of the properties just mentioned. The surface coating can also be a nanocoating, which forms a nanostructured surface. More secure and firmer securing as described above can be achieved in this way too. In particular, a surface coating can optionally also be electrically non-conductive, i.e. can have insulating properties. This can be considerably relevant to safety since possible burns due to electrical currents and heating on the skin of the patient can thereby be effectively avoided. The securing device can optionally therefore in particular also be used if electrical conductivity of the securing element or of individual components is not desired.

It is also possible for the skin piercing element to be hollow on the inside. As a result, it can be filled with a sticky material, for example with a fibrin adhesive or another biocompatible adhesive. It is thereby possible to fasten the securing device even more securely to the skin.

It is particularly advantageous if the entire securing device, or at least parts thereof, is made of antimicrobial material that repels dirt, blood and water, or is coated with such a material.

As a result of the configuration according to the invention of the securing device, it is likewise possible to fasten not only catheters but also other tubes and drains to the skin of the patient. Moreover, it is no longer necessary to shave the skin around the puncture site or around the point of entry of the catheter into the skin. Microlesions, which can serve as ports of entry for microorganisms, are avoided in this way. The clinical significance of the securing device according to the invention is considerable. For example, it can also be used whenever different tissues or tissue layers have to be secured in relation to one another, for example within the scope of surgical interventions or transplants, e.g. skin transplants. The securing device according to the invention can also be used in other, non-medical, regions.

As a result of the skin piercing element, which can be designed as fine skin clips with a defined length, it is moreover very unlikely for deeper structures beneath the skin to suffer damage. This can indeed happen when catheters are sutured to the skin in the customary manner. As a result of the securing device according to the invention, a catheter can be secured potentially more easily, more securely, more effectively and more efficiently.

In the context of the present invention, the indefinite article “a” or “an” is not to be understood as meaning a number. For example, if mention is made of a component, this is to be interpreted in the sense of “at least one component”. Insofar as angle specifications are given in degrees, they refer to a circular dimension of 360 degrees (360°).

The invention is explained in more detail below on the basis of exemplary embodiments and with use of drawings, in which:

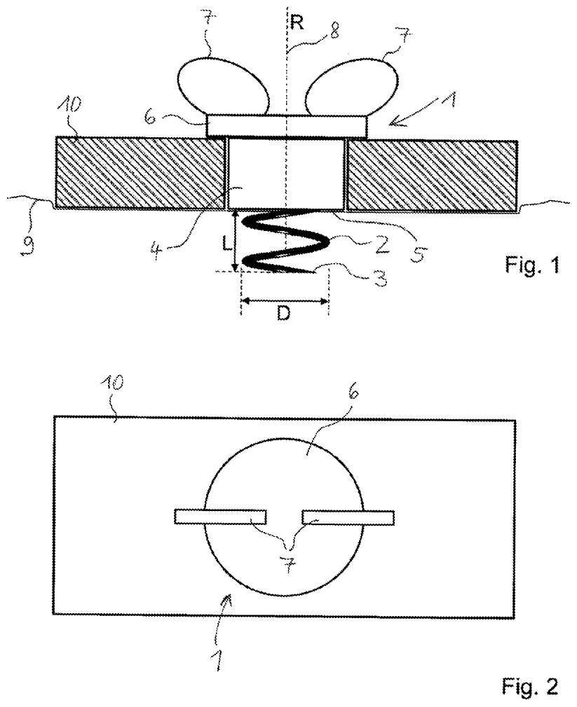

FIG. 1 shows an assembly comprising a retaining element and a securing device in a lateral sectional view,

FIG. 2 shows the assembly according to FIG. 1 in a top view,

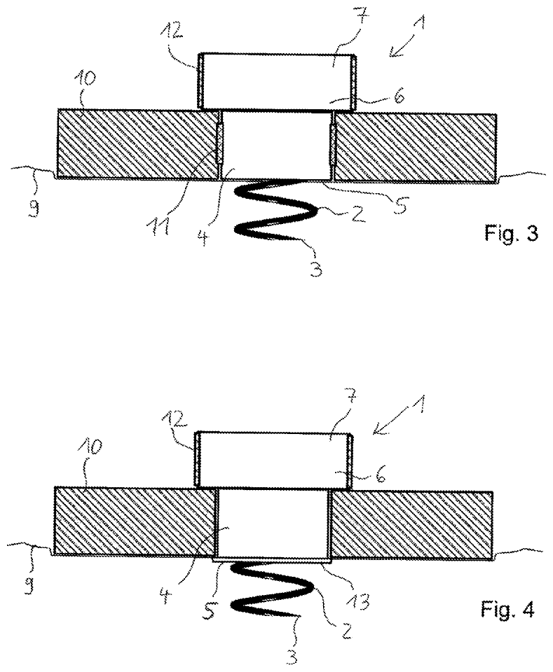

FIGS. 3-5 show further embodiments of an assembly comprising a retaining element and a securing device, in each case in lateral sectional views,

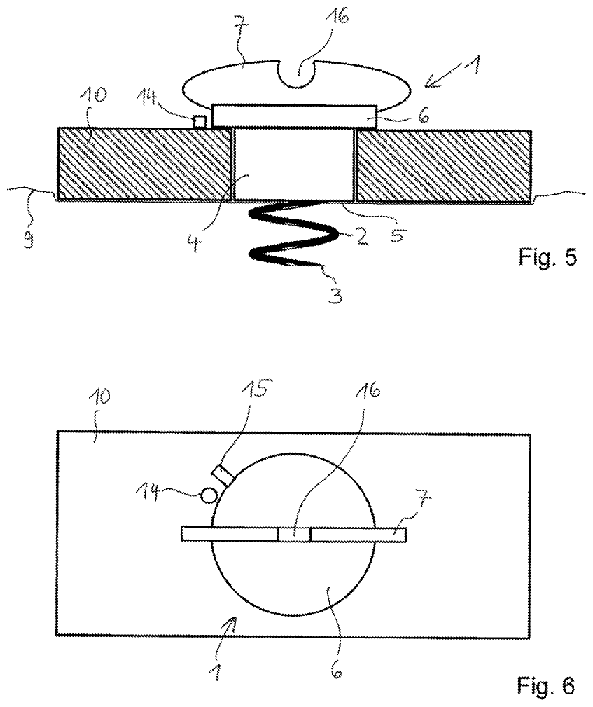

FIG. 6 shows the assembly according to FIG. 5 in a top view,

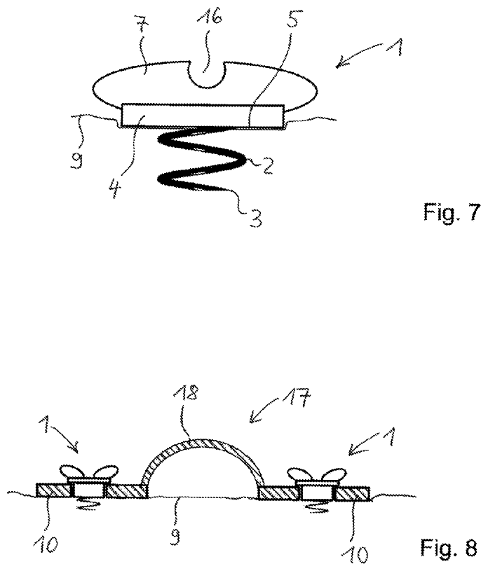

FIG. 7 shows a securing device in side view, and

FIG. 8 shows an assembly comprising retaining elements and securing devices in a lateral sectional view.

The reference signs used in the drawings have the following assignment:
1 Securing device
2 Skin piercing element
3 Piercing tip
4 Coupling portion
5 Skin contact surface
6 Securing portion
7 Actuating element
8 Axis of rotation
9 Skin
10 Retaining element
11 Connecting element
12 Friction-increasing structure
13 Connecting element
14 Travel limiting element
15 Stop
16 Securing portion
17 Securing bridge
18 Bridge region

The assembly illustrated in FIGS. 1 and 2 comprises a retaining element 10 and a securing device 1. The retaining element 10 is fastened to the skin 9 of a living being by means of the securing device 1.

The securing device has a skin piercing element 2 which has a piercing tip 3 at the free end. The skin piercing element 2 may be configured, as illustrated, in the form of a spiral spring. The skin piercing element 2 is connected to a coupling portion 4 of the securing device 1. The skin piercing element 2 is coupled here to the coupling portion 4 so as to be nonrotatable about an axis of rotation 8. The coupling portion 4 is adjoined on the side facing away from the skin piercing element 2 by a securing portion 6 of the securing device 1. The securing portion 6 protrudes over the coupling portion 4 in the circumferential direction, in a similar manner to a screw head in a commercially available screw. One or more manual actuating elements 7 of the securing device 1 are fastened to the securing portion 6.

The securing device 1 can be screwed by means of the skin piercing element 2 into the skin 9 by rotation about the axis of rotation 8 or, in the illustration of FIG. 1, is already screwed in there. The skin piercing element 2 is located with its region protruding from the coupling portion 4 completely inside the skin 9. The securing device 1 rests here on the skin with a skin contact surface 5. The skin contact surface 5 can be arranged, for example, on that side of the coupling portion 4 which faces the skin 9.

The skin piercing element 2 can have a length L, for example in the range of 1 to 10 mm, or in the range of 1.5 to 4 mm, in the direction of longitudinal extent along the axis of rotation 8. For example, a length L of approx. 2 mm is advantageous. The diameter D of the skin piercing element 2 can be selected to be smaller or greater, depending on the intended use, and depending on in which type of living being (human, animal) the securing device can be used. For example, the diameter D can be up to 25 mm. For uses in humans, for example, a diameter D in the range of 2 to 10 mm, or 3 to 8 mm, is advantageous.

The material diameter of the skin piercing element in the form of a spiral spring, i.e. the diameter of the wire or other material used for this purpose, can be, for example, in the range of 1 to 2 mm.

FIG. 3 shows an alternative embodiment of the securing device 1 in a similar arrangement as in FIG. 1. The securing device 1 has a different shaping of the securing portion 6 and of the actuating element 7, which are combined to form a common portion. The portions 6, 7 can be configured, for example, similarly to a cylindrical, hemispherical or circumferentially polygonal body. There can be a friction-increasing structure 12, for example a roughened surface, longitudinal grooves, a knurling or the like, on the outer circumference of this body, which forms the securing portion 6 and the actuating element 7.

In addition, the securing element 1 according to FIG. 3 has a connecting element 11 which is arranged on the outer circumference of the coupling portion 4. In this way, the securing device 1 is fastened captively to the retaining element 10. The connecting element 11 can be configured, for example, as an elastomer coating, external thread, protruding collar or the like. If the connecting element 11 is configured as an external thread, it is advantageous if the holding element 10 has an internal thread matched thereto on the inner circumference of the cutout. If the connecting element 11 is configured as a protruding collar, it is advantageous if the retaining element 10 has an internal groove matched thereto on the inner circumference of the cutout.

FIG. 4 shows an alternative embodiment of the securing device 1 that differs from the embodiment of FIG. 3 in that a different connecting element 13 is provided. In this case, the connecting element 13 is arranged at that end of the coupling portion 4 which faces the skin 9, specifically as an end-side collar protruding circumferentially over the coupling portion 4. This embodiment has the advantage that the retaining element 10 does not have to be specially adapted for securing the connecting element 13. Instead, the securing device 1 can simply be pushed through the cutout of the retaining element 10, with the connecting element 13 yielding somewhat here because of a flexible configuration and rebounding again after the coupling portion 4 is pushed through the cutout. This can be realized, for example, by the coupling portion 4 together with the connecting element 13 being configured as a plastic component, e.g. made from a plastic with sufficient flexibility in order to enable the connecting element 13 to be passed through the cutout. For example, the coupling portion 4 can be produced as a plastic injection molded component, wherein the skin piercing element 2 can be immediately injected there in the injecting procedure and can therefore be anchored therein. The remaining regions of the securing device 1, in particular the securing portion 6 and the actuating element 7, can likewise be produced integrally with the coupling portion 4 in a plastic injection molding process.

FIGS. 5 and 6 show a further embodiment of a securing device 1. In this case, the actuating element 7 is configured as a web which is continuous in the transverse direction and which has a cutout 16. The cutout 16 can be used as a further securing portion of the securing device 1, for example by a tube being firmly clamped there.

As FIGS. 5 and 6 show, the securing device 1 can additionally have a stop 15. The stop 15 can interact with at least one travel limiting element 14 which is formed on the retaining element 10. By interaction of the stop 15 with the travel limiting element 14, the maximum angle of rotation of the securing device 1 is limited. If the stop 15 comes to rest against the travel limiting element 14, the securing device 1 can no longer be rotated further in the respective direction of rotation.

A plurality of travel limiting elements 14, e.g. at least two travel limiting elements, can also be provided. They can be differently shaped and operable (e.g. can be pushed down). They can in particular also have the function of securing the position of the skin piercing element 2 in the securing state (“locking function”).

However, the travel limiting element 14 can also be configured here in the form in which it is connected to the retaining element 10 via a spiral spring. The spiral spring can be attached here recessed in a cylindrical cutout/a hole in the retaining element 10. It is thereby possible to actively push the travel limiting element 14 in the direction of the retaining element 10 and therefore to temporarily actively stop the action of the travel limiting element 14 in order nevertheless to be able to actively rotate the stop 15 via the travel limiting element 14. The travel limiting element 14 itself can be configured, for example, in the form of a ram or a ramp, but other forms are also conceivable.

The arrangement can have a depth scale at which the penetration depth of the skin piercing element 2 into the skin 9 can be read in accordance with the respective angle of rotation of the securing device 1 about the axis of rotation 8 in relation to the retaining element 10. The depth scale can be arranged, for example, on the surface of the retaining element 10 facing away from the skin 9. The depth scale can be configured here in multicolored form, for example even in the colors of a traffic light.

FIG. 7 shows an embodiment of a securing device 1 which is not configured for interaction with a retaining element 10, as described previously. The securing device 1 has merely the skin piercing element 2, a coupling portion 4 and a manual actuating element 7. The manual actuating element 7 can also be formed here with the coupling portion 4 as one component, similarly to the securing portion with the actuating element 7 in the embodiments of FIGS. 3 and 4.

The coupling element 4 or, if the latter is configured as a component with the actuating portion 7, said common component, in turn has the skin contact surface 5 on the side facing the skin 9. In this case, the cutout 16 to which, for example, a tube or another article can be fastened, is provided on the actuating element 7 as securing portion.

Said securing portion 16, which is illustrated as a cutout in the examples of FIGS. 5 to 7, may also be configured differently, for example as a pivotable or foldable element which can be pivoted or folded onto the actuating element 7 in order to secure the article that is to be secured on the securing device 1. This also includes the possibility that a type of cover can be pivoted via the securing portion 16 in order, for example, to even more securely secure a catheter tube in this fixing portion 16 if it is already in said securing portion 16.

FIG. 8 shows an assembly in which a securing bridge 17 is secured by two securing devices 1, which can be configured according to the previously explained type, to the skin 9. The securing bridge 17 has, for example, two retaining elements 10 of the previously described type. The retaining elements 10 are connected to one another via a bridge region 18, which can be, for example, curved or angled. The securing bridge 17 is therefore configured similarly to a hose clip. An article that is to be secured and that can be secured to the skin via the securing bridge 17 can be arranged below the bridge region 18. However, the bridge region 18 can also be deformable in all directions. As soon as the article that is to be secured is arranged under the bridge region 18, the latter can be pushed in the direction of the article that is to be secured. The bridge region 18 can therefore be in contact in a securing manner with the article to be secured or can at least partially surround said article with contact. The entire bridge region 18 or that side of the bridge region 18 which faces the article that is to be secured can additionally have here special material properties, such as a roughened surface, an increased frictional resistance or sticky properties for better securing of the article that is to be secured, or can be provided with a coating which has said properties.

1. A securing device designed for securing a medical, cosmetic, decorative or other article to skin of a living being, comprising:
at least one skin piercing element is designed to penetrate the skin of the living being, wherein the at least one skin piercing element comprises a piercing tip; and
a securing device comprising at least one securing portion coupled to the at least one skin piercing element, wherein the securing device is designed for securing the medical, cosmetic, decorative or other article to be the securing device,
wherein the securing device is designed for securing the skin piercing element in the skin of the living being by a rotational movement of the securing device, wherein an axis of rotation of the securing device runs orthogonally to a surface of the skin of the living being.

2. The securing device as claimed in claim 1, wherein the securing device comprises a manual actuating element at which the securing device is grippable manually and a rotational movement for securing the securing device on the skin of the living being is exertable on the securing device.

3. The securing device as claimed in claim 2 wherein the at least one securing portion is arranged on the manual actuating element.

4. The securing device as claimed claim 1 wherein the securing device comprises a skin contact surface which comes into contact with the surface of the skin of the living being when the securing device is secured on the skin of the living being.

5. The securing device as claimed in claim 4, wherein the at least one skin piercing element protrudes directly from the skin contact surface.

6. The securing device as claimed in claim 1 wherein the at least one skin piercing element is configured as a spiral spring.

7. The securing device as claimed in claim 6, wherein the spiral spring comprises spiral spring turns which are spaced apart from one another in a direction of a longitudinal axis of the at least one skin piercing element.

8. The securing device as claimed in claim 1 wherein the securing device comprises at least one stop by which a maximum angle of rotation of the securing device about the axis of rotation is limited.

9. The securing device as claimed in claim 6 wherein a main piercing element of the spiral spring has a pitch of less than 3 mm/360°.

10. An assembly, comprising:
a retaining element; and
at least one securing device as claimed in claim 1,
wherein the retaining element has at least one cutout through which the at least one securing device is passable at least together with the at least one skin piercing element,
wherein the retaining element is securable on the skin of the living being by the at least one securing device by the at least one skin piercing element that has been guided through the at least one cutout being secured in the skin of the living being by a rotational movement of the at least one securing device,
wherein the axis of rotation of the at least one securing device runs orthogonally to the surface of the skin of the living being.

11. The assembly as claimed in claim 10, wherein the at least one securing device has a coupling portion arranged in a longitudinal direction between the at least one securing portion and the at least one skin piercing element.

12. The assembly as claimed in claim 10, wherein the coupling portion is guidable into the cutout and is mounted rotatably in the cutout.

13. The assembly as claimed in claim 11 wherein the coupling portion directly adjoins the at least one skin piercing element in the longitudinal direction.

14. The assembly as claimed in claim 11 wherein the at least one securing portion or a further securing portion of the at least one securing device protrudes over the coupling portion in the circumferential direction.

15. The assembly as claimed in claim 11 wherein the coupling portion has, on an outer circumference, at least one connecting element via which the at least one securing device is fastenable to the retaining element and is mountable rotatably on the retaining element.

16. The assembly as claimed in claim 15 wherein the retaining element has a fastening element which is complementary to the at least one connecting element and which is bringable into engagement with the at least one connecting element of the securing device in order to fasten the securing device to the retaining element and to mount the securing device rotatably on the retaining element.

17. The securing device as claimed in claim 1 wherein the securing device has at least one electrical insulating element which prevents an electrical current flow between the securing device and an article secured on the at least one securing portion and the skin of the living being.
